(19) United States
(12) Patent Application Publication
(10) Pub. No.: US 2010/0036261 A1
(43) Pub. Date: Feb. 11, 2010

(54) METHOD AND DEVICE FOR IMAGE GUIDED SURGERY

(75) Inventors: Irving WEINBERG, Bethesda, MD (US); Pavel Stepanov, North Potomac, MD (US)

Correspondence Address: BARNES & THORNBURG LLP, 750-17TH STREET NW, SUITE 900, WASHINGTON, DC, 20006-4675, US

(73) Assignee: WEINBERG MEDICAL PHYSICS LLC, Bethesda, MD (US)

(21) Appl. No.: 12/535,236

(22) Filed: Aug. 4, 2009

Related U.S. Application Data
(60) Provisional application No. 61/086,402, filed on Aug. 5, 2008.

(51) Int. Cl. A61B 6/00

(52) U.S. Cl. 600/476

(57) ABSTRACT

A fluorometer includes a light source to generate excitatory light toward a tissue, the tissue generating fluorescent light in response to the excitatory light. The fluorometer also includes a light sensor to receive the fluorescent light and generate a digital signal. A processor is connected to the light sensor to receive the digital signal and generate a digital image, and a display displays the digital image. The tissue generates fluorescent light as a result of excitation of at least one intrinsic tissue metabolic product. A method for distinguishing between viable and non-viable tissue using the fluorometer also is described.

### CROSS-REFERENCE TO RELATED APPLICATION(S)

This is a United States Non-Provisional patent application that relies for priority on U.S. Provisional Patent Application No. 61/086,402, filed on Aug. 5, 2008, the contents of which are incorporated herein by reference.

### BACKGROUND OF THE INVENTION

The present invention involves an apparatus and a method that provides intraoperative imaging feedback to permit a podiatrist and/or surgeon to determine the margins between healthy and unhealthy tissue. More specifically, the present invention provides an apparatus and a method that permits imaging of tissue so that a surgeon may determine the margins between healthy and unhealthy tissue, especially in circumstances where the unhealthy tissue is to be removed surgically.

### DESCRIPTION OF THE RELATED ART

Generally speaking, diabetic patients are prone to a number of maladies associated with diabetes. Since diabetes results in a generally elevated glucose concentration in the patients blood, the patient's circulatory system is less efficient in delivering oxygen to tissues, especially within the patient's extremities. The extremities include the patient's hands and, more commonly, the patient's feet.

In more severe cases, one unfortunate consequence of diabetes is the necrosis (or death) of tissue at the patient's extremities. When the tissue in a diabetic's feet becomes necrotic, it becomes necessary to remove the necrotic portions, leaving the healthy tissue to remain.

When resecting or removing tissue, it is often difficult for a surgeon to delineate the margins between healthy and unhealthy tissue. This may lead, in some cases, to the removal of too much tissue from the affected area. In other cases, this difficulty may lead to the removal of too little tissue, which may necessitate subsequent surgeries.

More specifically, from a medical standpoint, many diabetic patients suffer from one or more factors (e.g., reduced blood flow, impaired immune response, neuropathies) that predispose them to osteomyelitis.

Due to the effects of diabetes on the person, about a quarter of American diabetic patients will have foot problems. Among hospital admissions for diabetes, 20% are for foot osteomyelitis.

Antibiotic treatment of osteomyelitis is long, expensive and often ineffective, leading to the aggressive use of preventive surgery at an early stage.

As noted above, it is the goal of the surgeon to resect as little healthy tissue as possible, in order to preserve normal gait and stance.

Given this goal, there is diversity of opinion within the diabetic care community as to appropriate algorithms for minimizing resection volumes. At many sites, surgical minimization is accomplished with an iterative approach, in which patients reside in hospital beds while specimens are examined histologically. One goal with this proposal is to provide surgeons and podiatrists with immediate feedback as to tissue viability and bacterial load, thereby allowing procedures to be accomplished more conservatively and confidently, while decreasing the duration of hospital stays.

Generally speaking, the most common surgical guidance application is the use of a priori data sets (e.g., Positron Emission Tomography (“PET”) scans and x-ray films). Although traditionally these scans are viewed by surgeons prior to the procedure, it is possible to register the a priori data sets to position sensors for a road map view during surgery. As should be apparent to those skilled in the art, one drawback to a priori methods is that the road maps are not updated during surgery. In other words, images taken before surgery may not reflect the condition of the patient during surgery. For example, an x-ray film developed a day before a surgery may not reflect the current condition of the patient due to the passage of time.

Intraoperative anatomic images have long been available for orthopedic and breast surgical procedures (e.g., using C-arm x-ray configurations and ultrasound). However, as should be appreciated by those skilled in the art, bringing functional images (i.e., that provide information about physiology or biochemistry) into the operative suite is challenging.

Alternatively, it is possible to perform surgery within an MRI or PET scanner bore, or to rapidly move a patient into the bore in the course of surgery, but these are costly solutions, which are not available to the large majority of patients, and which are inconsistent with many surgical procedures due to access limitations.

Hand-held radiation-counting devices (“probes”) also have been used in surgical oncology for intraoperative procedures in which patients received with tumor-avid radiotracers preoperatively. Hand-held gamma cameras have been promoted for clinical settings with high target-to-background contrast, such as localizations of sentinel nodes and removal of osteoid osteoma nidi.

Transferring technology from oncology to intraoperative wound care requires consideration of relevant clinical requirements. Except for high-contrast scenarios such as osteoid osteomas, radiotracer bone scans (e.g., with Tc-99 MDP) require long acquisition times to produce high confident images of surgical margins. Specifically, the radiotracer needs to be administered to the patient for a long period of time before the surgical procedure to assure that the patient's tissue has retained sufficient quantities of the radiotracer for accurate detection.

In addition, it is known that non-imaging probes are useful in detection of “hot spots” encountered in sentinel node procedures, but they are less useful in defining the limits of normal tissue.

As should be appreciated by those skilled in the art, clinicians outside the nuclear medicine department are sometimes averse to the radiation exposure from such procedures. This adversity impedes market penetration of radiation-based apparatuses, techniques, and methodologies.

Infrared imaging devices that are able to detect oxygenated blood flow have been developed to assess long-term healing of diabetic foot ulcers. For validation and clinical purposes, it is contemplated that this same technique may be employed to identify the difference between diseased and normal tissue, even after the tissue has been resected. However, it is contemplated that this technique would not be likely to succeed because it relies on blood flow as the source signal—blood flow is not available in every circumstance. In addition, the OxyVu instrumentation marketed by a company called HyperMed, Inc. in Burlington, Mass., since it relies on blood flow as the source of signal, would not be a good candidate for the reasons enumerated above.

As should be appreciated by those skilled in the art, many exogenous contrast materials are available which are optically-active, but only a few of these can be administered safely to a patient prior to surgery. For example, tetracycline, administered to patients for months prior to surgery, has been used in some studies as a marker for cell viability. Intraoperative ultraviolet lights have been used to detect tetracycline fluorescence in order to confirm that margins are clear of necrotic tissue. One drawback of this technique is the long loading time of the contrast material, which may lead to inaccurate identification if the patient's condition changes within weeks prior to surgery. Another drawback is that some patients may be allergic to tetracycline, or may harbor infectious organisms resistant to this antibiotic.

As may be appreciated from the foregoing, there remains a need for devices and techniques that permit a physician and/or a surgeon to differentiate between healthy and unhealthy tissues during a surgical operation.

### SUMMARY OF THE INVENTION

It is, therefore, one aspect of the present invention to provide an apparatus that permits a physician and/or surgeon to identify the margins between healthy and unhealthy tissue during a surgical procedure.

It is another aspect of the invention to provide a method for identifying the margins between healthy and unhealthy tissue during a surgical procedure.

As will be made apparent from the discussion that follows, aspects of the invention are attained by providing immediate, intraoperative imaging feedback to podiatrists and surgeons as to whether surgical margins are clear of involvement, and whether remaining tissues are healthy.

Often, patients reside in hospital beds while pathological assessment of tissue specimens is pending, adding to overall care costs and exposing patients to potential nosocomial infection. A rapid and reliable intraoperative method of assessing surgical margins could potentially reduce hospital stays. Moreover, providing additional confidence might allow surgeons and podiatrists to be more conservative in their resections, thereby improving patients' quality of life.

Unlike prior imaging approaches that employed exogenous contrast materials (e.g., tetracycline, labeled radiotracers) the present invention exploits the availability of endogenous markers (e.g., NADH, which is the reduced form of nicotinamide adenine dinucleotide (“NAD”)) to rapidly assess tissue viability.

While contemplated to be employed for purposes of identifying the margins between healthy and unhealthy tissue in diabetic patients, the present invention may be applied to a large variety of circumstances, including, for example, decubitus ulcer debridement and other wound care challenges.

It is, therefore, an aspect of the present invention to provide a fluorometer that includes a light source to generate excitatory light (i.e., ultraviolet) toward a tissue, the tissue generating fluorescent light in response to the excitatory light, a light sensor to receive the fluorescent light and generate a digital signal, a filter interposed between the light source and the light sensor, wherein the filter optimizes the fluorescent light impingent on the light sensor, thereby optimizing the digital signal generated by the light sensor, a processor connected to the light sensor to receive the digital signal and generate a digital image, and a display to display the digital image.

In one contemplated embodiment, the tissue generates fluorescent light as a result of excitation of at least one intrinsic tissue metabolic product, such as NADH.

The present invention provides for a fluorometer where the digital image includes information permitting differentiation between viable and non-viable tissue.

In a contemplated embodiment, the light source comprises a light emitting diode.

In another contemplated embodiment, the filter is a spectral filter. Alternatively, the acquisition of the fluorescent image may be delayed until after the excitatory pulse of ultraviolet light has decayed.

For the present invention, it is contemplated that the light sensor is a digital camera.

In another contemplated embodiment, at least one lens is disposed between the filter and the light sensor to focus the fluorescent light on the light sensor.

The present invention also contemplates a method for distinguishing between viable and non-viable tissue. The method includes generating excitatory (i.e., ultraviolet) light by a light source, illuminating a tissue with the excitatory light, whereupon the tissue responds by generating a fluorescent light, sensing the fluorescent light by a light sensor, generating a digital signal by the light sensor from the fluorescent light, filtering the excitatory light and the fluorescent light via a filter interposed between the light source and the light sensor to optimize the fluorescent light impingent on the light sensor, thereby optimizing the digital signal generated by the light sensor, generating a digital image by a processor connected to the light sensor, and displaying the digital image on a display. The method relies on generation of fluorescent light by the tissue as a result of excitation of at least one intrinsic tissue metabolic product, such as NADH.

In another contemplated embodiment, the method includes focusing the fluorescent light on the light sensor by at least one lens disposed between the filter and the light sensor.

The digital image produced by the method may be used in a tissue debridement procedure.

Other aspects of the present invention will be made apparent from the discussion that follows.

### BRIEF DESCRIPTION OF THE DRAWINGS

The invention will now be described in connection with the illustrations appended hereto, in which:

FIG. 1 is a schematic overview of one contemplated embodiment of the fluorometer apparatus of the present invention; and

FIG. 2 is a flow chart illustration providing one contemplated embodiment of a method of the present invention for distinguishing between viable and non-viable tissue.

### DETAILED DESCRIPTION OF EMBODIMENT(S) OF THE INVENTION

The present invention will now be described in connection with one or more embodiments. The discussion of specific embodiments, however, is not intended to be limiting of the invention. To the contrary, the selected embodiments are intended to be exemplary of the broad scope of the present invention. As should be appreciated by those skilled in the art, there are numerous variations and equivalents that may be employed without departing from the present invention. Those embodiments and variations are intended to be encompassed by the present invention.

While the present invention is described primarily in connection with the identification of healthy and unhealthy tissue in the context of a diabetic patient, it is contemplated that the present invention will be applicable to a wide variety of circumstances.

As indicated above, fluorescence is exhibited naturally by many organisms, primarily from NADH. NADH is component of the Krebs cycle, with broad application to biological assays.

In most aerobic cells, NADH is chemically oxidized to NAD, which is not fluorescent. NADH is, therefore, a reasonable candidate as an endogenous signature of non-viable bone tissue and for active infectious organisms.

Fluorometers have been constructed using active pixel sensors (“APS”) as light detectors, and by using spectral filters to shield the APS photodetectors from the light generated by the excitation source (e.g., ultraviolet light-emitting diodes (“UV LEDs”)). Such filters can have a long-pass characteristic with 400 nm cut-on frequency and 60-dB rejection in the UV region, suitable for imaging UV-excitable fluorophores with emission wavelengths above 400 nm. NADH fluorescence is centered at 450 nm. Other filters also may be employed, as should be appreciated by those skilled in the art.

The scientific literature suggests that fluorescence from recently resected bone will exhibit initially a low (but non-zero) signal, followed by high signal due to anoxia. Forensic studies suggest that the NADH fluorescence signal from resected bone tissues will very slowly decay (i.e., over tens to hundreds of years).

In the present invention, the detection within images of bony regions with strong NADH-derived fluorescence serves as a marker for tissue non-viability, which the surgeon can therefore remove.

It is understood that since certain microbes also exhibit NADH-derived fluorescence, it may be necessary for the surgeon to clean the bony surface in order to verify that the abnormal signal is not an artifact. It is understood that certain techniques (e.g., optical coherence tomography) may be applied in order to increase signal to noise and provide images of deep tissues.

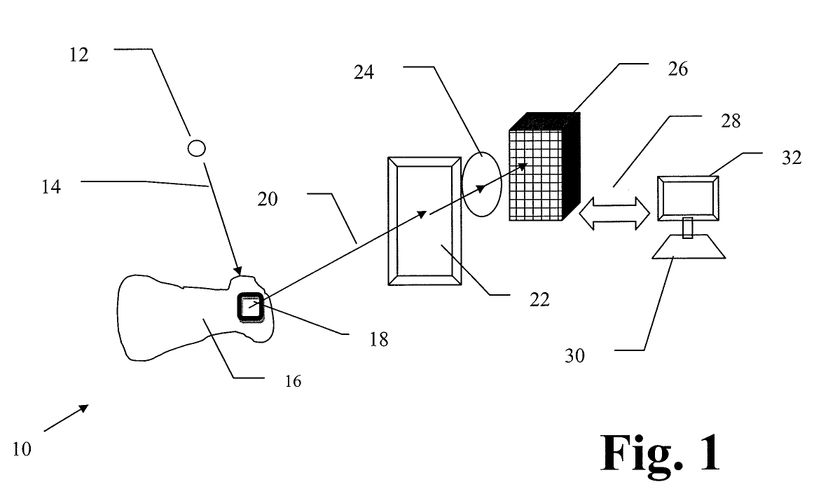

As illustrated in FIG. 1, the fluorometer apparatus 10 of the present invention includes a light source 12, which emits excitatory light 14. In one contemplated embodiment, the light source 12 emits ultraviolet light 14. To this end, it is noted that the terms “excitatory light” and “ultraviolet light” are used interchangeably herein. While the present invention is intended to encompass light with wavelengths outside of the ultraviolet spectrum, the wavelengths that encompass the ultraviolet portion of the electromagnetic spectrum are believed to generate optimal fluorescence from an illuminated target, such as tissue 16.

As illustrated in FIG. 1, the ultraviolet light 14 is directed toward the tissue 16. The ultraviolet light 14 induces fluorescence in the tissue 16 due to the presence of NADH, as discussed above. As illustrated in FIG. 1, a portion of the tissue 18 that includes NADH emits fluorescent light 20. The portion of the tissue 18 that exhibits fluorescence from NADH, therefore, is non-viable or unhealthy tissue.

The light source 12 may be any type of light source that emits ultraviolet light so that the NADH in the tissue 16 fluoresces. As may be appreciated by those skilled in the art, the light source 12 may be an incandescent bulb, a fluorescent bulb, or a light emitting diode (“LED”). The light source 12 also may be any of a number of other sources of ultraviolet light, including light that has been filtered from a source that emits a plurality of different wavelengths. In addition, it is contemplated that the light source 12 may provide a coherent beam of ultraviolet light, as is possible with a laser emitting diode, for example. In still another contemplated embodiment, light 14 from the light source 12 may be applied to the tissue 16 via one or more optical fibers. The light 14 also may be concentrated via one or more lenses before application to the tissue 16 without departing from the scope of the present invention. As should be apparent, the exact nature and construction of the light source 12 is not critical to operation of the present invention.

While the exact nature and construction of the light source 12 is not critical to operation of the present invention, it is contemplated that the light source will include one or more LEDs. LEDs are preferred for use in the present invention due to their small size, which permits the light source 12 to be placed close to the tissue 16, if needed or desired.

With respect to the light source 12, it is contemplated that the light source 12 will be constructed so that the light source 12 may emit ultraviolet light of varying wavelengths and energies. To this end, the light source 12 may be constructed to permit the user to change the wavelength(s) of the emitted light. Alternatively, the light source 12 may be a combination of several emitters of different wavelengths, as necessary or as desired. For example, the light source 12 may combine several LEDs together, each of which emits light with wavelengths slightly different from the next.

As should be appreciated by those skilled in the art, ultraviolet light typically is defined as light with a wavelength range of 10 to 400 nm and with energies of 3 to 124 eV. This definition of ultraviolet light, however, should not be considered to be limiting of the present invention as it is anticipated that longer wavelength light in the violet band, for example, may also be employed to incite fluorescence of a suitable substance, such as NADH. As discussed above, the present invention is intended to encompass any wavelength of excitatory light that falls outside of the accepted range of wavelengths for ultraviolet light.

The fluorescent light 20 emitted by the tissue portion 18 is expected to be at or near a wavelength of 450 nm, corresponding to a principal portion of the emission spectrum of NADH, as discussed above. The fluorescent light 20 is first passed through a filter 22. The filter 22 is provided to eliminate any of the ultraviolet light 14 impingent thereon from the light source 12.

As should be appreciated by those skilled in the art, the filter 22 may not be required, depending upon the construction of the light sensor 26, described in greater detail below. For example, the light sensor 26 may be designed to detect the fluorescent light 20 only within a suitable range of wavelengths, for example, near the 450 nm for the light emitted by excited NADH.

Alternatively, the filter 22 may be constructed as a plurality of filters or a variable wavelength filter, as required or desired. Variability for the filter 22 may be desired, for example, to change the wavelength of the fluorescent light 20 that is impingent on the light sensor 26. This may be required or desired depending upon the tissue 16 being illuminated or depending upon other factors that should be appreciated by those skilled in the art.

The fluorescent light 20 passes through the filter 22, whereupon it enters a lens 24. The lens 24 concentrates and focuses the fluorescent light 20 onto an image forming sensor 26. The image forming sensor 26 is sensitive to the fluorescence but relatively insensitive to the excitatory light 14.

As should be appreciated by those skilled in the art, the lens 24 may be a simple lens, a compound lens, a single lens, a series of lenses, or the like. The exact composition of the lens 24 is not critical for operation of the present invention, as should be appreciated by those skilled in the art.

Separately, it is contemplated that a lens 24 may not be required at all. In other words, the lens 24 may be omitted completely from the fluorometer 10 without departing from the scope of the present invention.

The light sensor 26 may be one of any of a number of different types of light-sensing components. For example, the light sensor 26 may be a digital camera. Further still, the light sensor 26 may be a complimentary metal oxide semiconductor (“CMOS”) device. Alternatively, the light sensor 26 may be a charge-coupled device (“CCD”). Since CMOS and CCD devices are typically incorporated into digital cameras, the term “digital camera” is intended to encompass any electronic device that generates a digital signal that may be assembled into a digital image. A great variety of different sensing elements may be selected for the light sensor 26, as should be appreciated by those skilled in the art. The precise element selected or employed is not critical to operation of the fluorometer 10 of the present invention.

In the illustrated embodiment, the fluorescent light 20 passes through the lens 24 and impinges upon the light sensor 26. The impingent fluorescent light 20 causes the sensor 26 to generate one or more electrical signals that are provided, via a communication link 28, to a processor 30. The processor 30 includes one or more programs that permit analysis of the electrical signals.

Alternatively, the filter 22 may be functionally replaced, or be supplemented by, a time delay mechanism with respect to data acquisition. In this embodiment, the initiation of conversion of electrical signals generated by light sensor 26 into data (i.e., data acquisition) by processor 30 may be delayed by the light sensor 26 so that the ultraviolet light 14 from the light source 12 is not captured, whereas the fluorescent light 20 from the tissue portion 18 is captured. This approach is contemplated to be particularly applicable in a pulsed light (or strobed) environment. In this example, immediately after completion of a pulse of ultraviolet light 14, the processor 30 does not accept the electrical signals generated by the light sensor 26. Only after the tissue portion 18 generates fluorescent light 20 does the processor 30 capture the electrical signals generated by the light sensor 26. In this fashion, any signals generated by the light sensor 26 in response to captured ultraviolet light 14 may be ignored.

Alternatively, the delay may be introduced in the processing step implemented by processor 30.

As should be apparent to those skilled in the art, the light sensor 26 preferably is selected so that the light sensor 26 generates one or more digital signals. The digital signals indicate at least a magnitude of the fluorescent light 20 emitted from the tissue portion 18. Other parameters that may be detected may include the wavelength of the fluorescent light 20, as required or as desired.

The processor 30 may be provided with a display screen 32 that displays an image representative of the areas that emit the fluorescent light 20. The display may be akin to the display of a photograph. Naturally, to permit the surgeon and/or physician to appreciate the margin between the healthy and the unhealthy tissue, the image displayed on the screen 32 may be enhanced visually. The visual enhancement may be the result of algorithms applied to the original digital image by the processor 30. Since the digital image is shown on the display screen 32 in real time, the physician and/or surgeon may assess the current health status of the tissue 16, 18 during a resection or similar operation.

With continued reference to FIG. 1, the communication link 28 is intended to be any suitable one-, two-, or multi-way link between the sensor 26 and the processor 30. As should be appreciated by those skilled in the communication link 28 may include a plurality of links, as required or desired. Additionally, the communication link 28 may be wired or wireless.

As should be apparent from the foregoing, a user examines the fluorescent image to determine which areas fluoresce, corresponding at least in part to the local oxidation state of NADH. The user will then incorporate this information into his or her decision-making process as to whether to resect the specific region of tissue 18.

As may be appreciated, any number of excitatory light sources 12 and filters 22 may be employed as calibration tools in order to provide additional quantitative information that may be displayed in the image to the user. In addition, the camera 26 may be rendered relatively insensitive to the excitatory light 14 by the use of delayed camera triggering, so that the light 14 from the initial excitation is diminished by the time the camera 26 becomes active. Still further variations may be appreciated by those skilled in the art.

It is noted that the fluorometer 10 assists with diagnosis and treatment of a patient by providing a digital image for the user that distinguishes between viable (i.e., healthy) tissue and non-viable (i.e., non-healthy) tissue. This distinction assists the user to resect non-viable tissue. This distinction also may be relied upon during a tissue debridement procedure. This distinction also may be relied upon for purposes of analyzing tissue pathologies, among other medical procedures, as should be appreciated by those skilled in the art.

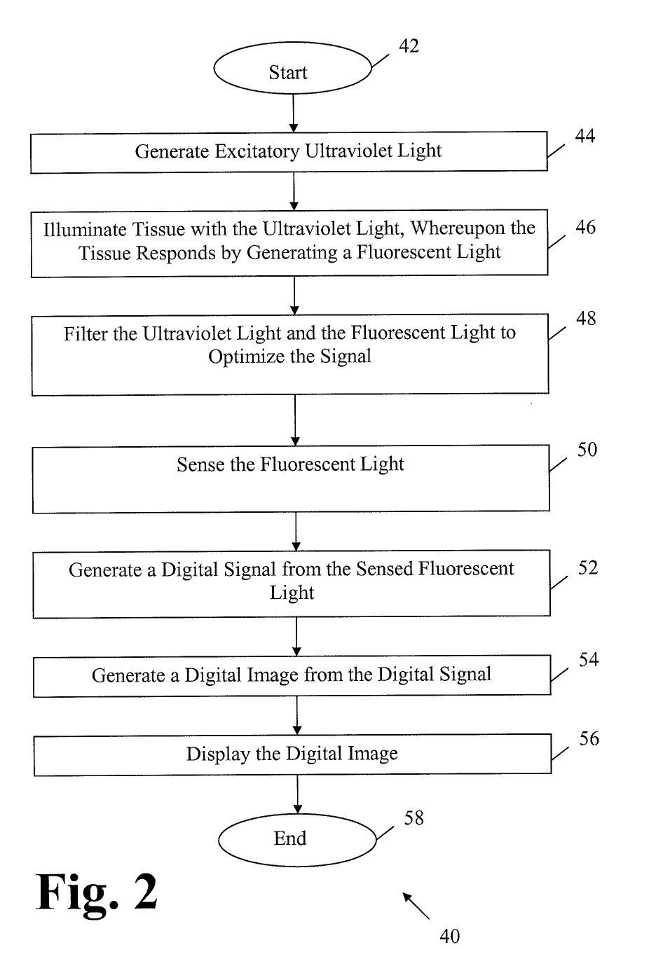

Next, with reference to FIG. 2, a method 40 of establishing a margin between healthy tissue and unhealthy tissue is provided. As noted above, when NADH is present in tissue 18, the NADH will fluoresce when exposed to ultraviolet light 14. The presence of NADH indicates that the tissue 18 is non-viable. As a result, tissue 16 that includes NAD is considered to be healthy tissue and will not fluoresce when exposed to ultraviolet light 14.

The method 40 includes several steps. The method begins at 42. The first step in the method 40 is designated 44. At 44, the method 40 generates excitatory (i.e., ultraviolet) light 14. The ultraviolet light 14 may be generated by the light source 12, which is discussed above. At 46, the method 40 illuminates a tissue 16 with the ultraviolet light 14, whereupon at least a portion of the tissue 18 responds by generating a fluorescent light 20.

The mixture of ultraviolet light 14 and emitted fluorescent light 20 is filtered in step 48, so that the influence of the ultraviolet light 14 is minimized. Subsequently, at 50, the method 40 senses the fluorescent light 20, such as by a light sensor 26, also discussed above. The filtering step 48 is optional.

The method 40 also includes the step of generating a digital signal through acquisition of electrical signals generated by the light sensor 26 from the fluorescent light 20, which step is designated at 52. This step may be delayed in order to provide an opportunity for the ultraviolet light 14 to decay.

The digital signal is provided to a processor 30 via a communication link 28. Thereupon, at 54, a digital image is generated from the digital signal. The digital image may be generated by a processor 30 connected to the light sensor 26, as discussed above. The digital image is displayed at step 56. The digital image may be displayed on a display 32, such as a computer monitor, as illustrated in FIG. 1. The method 40 ends at 58.

As indicated above, by the method 40, the tissue generates fluorescent light 20 as a result of excitation of at least one intrinsic tissue metabolic product. The intrinsic tissue metabolic product may be NADH, as discussed herein. Consequently, the method 40 generates the digital image such that the digital image includes information permitting differentiation between viable tissue 16 and non-viable tissue 18.

The method 40 may operate where the light source 12 is an LED, as discussed above. The filtering step 52 of the method 40 may be accomplished by a spectral filter. Alternatively, the filtering step 48 may be implemented such that the filter 22 introduces a time delay between the arrival of the ultraviolet light 20 and the fluorescent light 14 on the light sensor 26. As noted above, the light sensor 26 may be a digital camera.

Additionally, as discussed above, the method 40 may include a step of focusing the fluorescent light 20 on the light sensor 26 by at least one lens 24 disposed between the filter 22 and the light sensor 26.

Other embodiments of the present invention may be apparent to those skilled in the art based on the discussion herein and the illustrations appended hereto. Those variations and equivalents appreciated by those skilled in the art are intended to be encompassed by the present invention.

What is claimed is:

1. A fluorometer, comprising:
a light source to generate excitatory light toward a tissue, the tissue generating fluorescent light in response to the excitatory light;
a light sensor to receive the fluorescent light and generate a digital signal;
a processor connected to the light sensor to receive the digital signal and generate a digital image; and
a display to display the digital image,
wherein the tissue generates fluorescent light as a result of excitation of at least one intrinsic tissue metabolic product.

2. The fluorometer of claim 1, wherein the excitatory light is ultraviolet light.

3. The fluorometer of claim 1, wherein the at least one intrinsic tissue metabolic product is NADH.

4. The fluorometer of claim 1, wherein the digital image includes information permitting differentiation between viable and non-viable tissue.

5. The fluorometer of claim 1, wherein the light source comprises a light emitting diode.

6. The fluorometer of claim 1, further comprising:
a spectral filter interposed between the light source and the light sensor, wherein the filter optimizes the fluorescent light impingent on the light sensor by filtering out at least a portion of the excitatory light.

7. The fluorometer of claim 1, further comprising:
a filter that introduces a time delay in acquiring the digital signal from the light sensor until at least a portion of the excitatory light has decayed, thereby optimizing capture of the fluorescent light.

8. The fluorometer of claim 1, wherein the light sensor comprises a digital camera.

9. The fluorometer of claim 1, further comprising:
at least one lens disposed between the filter and the light sensor to focus the fluorescent light on the light sensor.

10. A method for distinguishing between viable and non-viable tissue, comprising:
generating excitatory light by a light source;
illuminating a tissue with the excitatory light, whereupon the tissue responds by generating a fluorescent light;
sensing the fluorescent light by a light sensor;
generating a digital signal by the light sensor from the fluorescent light;
generating a digital image by a processor connected to the light sensor; and
displaying the digital image on a display,
wherein the tissue generates fluorescent light as a result of excitation of at least one intrinsic tissue metabolic product.

11. The method of claim 10, wherein the excitatory light is ultraviolet light.

12. The method of claim 10, wherein the at least one intrinsic tissue metabolic product is NADH.

13. The method of claim 10, wherein the digital image includes information permitting differentiation between viable and non-viable tissue.

14. The method of claim 10, wherein the light source comprises a light emitting diode.

15. The method of claim 10, further comprising:
filtering, via a spectral filter, light impingent on the light sensor to optimize the fluorescent light impingent on the light sensor by filtering out at least a portion of the excitatory light.

16. The method of claim 10, further comprising:
introducing a time delay in acquiring the digital signal from the light sensor until at least a portion of the excitatory light has decayed, thereby optimizing capture of the fluorescent light.

17. The method of claim 16, wherein the time delay is introduced by the processor prior to step of generating the digital image.

18. The method of claim 10, wherein the light sensor comprises a digital camera.

19. The method of claim 10, further comprising:
focusing the fluorescent light on the light sensor by at least one lens disposed between the filter and the light sensor.

20. The method of claim 10, wherein the digital image is used in a tissue debridement procedure.
